(19) United States
(12) Patent Application Publication
(10) Pub. No.: US 2015/0306011 A1
(43) Pub. Date: Oct. 29, 2015

(54) METHODS FOR USING SOY PEPTIDES TO INHIBIT H3 ACETYLATION, REDUCE EXPRESSION OF HMG-COA REDUCTASE AND INCREASE LDL RECEPTOR AND SP1 EXPRESSION IN A MAMMAL

(71) Applicant: ALFREDO FLORES GALVEZ, West Sacramento, CA (US)

(72) Inventor: ALFREDO FLORES GALVEZ, West Sacramento, CA (US)

(21) Appl. No.: 14/261,080

(22) Filed: Apr. 24, 2014

(51) Int. Cl. A61K 8/64; A61Q 19/08

(52) U.S. Cl. CPC A61K 8/645; A61Q 19/08

(57) ABSTRACT

Controlled studies demonstrate that methods using soy related peptides inhibit H3 acetylation, reduce expression of HMG-CoA reductase and increase LDL receptor and Sp1 expression in mammals. The present disclosure is generally directed to using lunasin peptides and/or lunasin peptide derivatives to 1) inhibit H3 acetylation, 2) reduce expression of HMG-CoA reductase, 3) increase LDL receptor expression or 4) increase Sp1 expression in a mammal. In at least one exemplary embodiment of the present disclosure, an effective amount of lunasin peptides or lunasin peptide derivatives and one or more enzyme inhibitors is provided to a mammal to 1) inhibit H3 acetylation, 2) reduce expression of HMG-CoA reductase, 3) increase LDL receptor expression or 4) increase Sp1 expression in a mammal. Additionally, lunasin will protect against, prevent, or reduce: 1) the expression of Matrix metalloproteinase (MMP-1), 2) collagen breakdown, 3) photoaging and 4) the formation of skin wrinkles.

### RELATED APPLICATIONS

This application claims the benefit of priority to U.S. patent application Ser. No. 12/756,126, filed Apr. 7, 2010, which claims the benefit of priority to U.S. patent application Ser. No. 11/532,528 filed Sep. 16, 2006, and which also claims the benefit of priority to U.S. patent application Ser. No. 12/441,384, filed Mar. 14, 2009. U.S. patent application Ser. No. 12/441,384, filed Mar. 14, 2009, claims the benefit of priority to International Application Number PCT/US07/78584, filed Sep. 15, 2007, which claims priority to U.S. provisional No. 60/966,529, filed Sep. 16, 2006 (formerly U.S. patent application Ser. No. 11/532,526, filed Sep. 16, 2006) and claims priority to U.S. provisional No. 61/007,925, filed Jul. 17, 2007 (formerly U.S. patent application Ser. No. 11/879,249, filed Jul. 17, 2007.) All of the above listed applications are hereby incorporated by reference herein in their entireties.

### BACKGROUND

1. Technical Field

This disclosure relates generally to a class of peptides that provide mammals with a variety of health related benefits. More specifically, the present disclosure related to using soy peptides to inhibit H3 acetylation, reduce expression of HMG-CoA reductase, increase LDL receptor and Sp1 expression, protect against or prevent the expression of Matrix metalloproteinase (MMP-1), the breakdown of collagen, photoaging and the formation of skin wrinkles.

2. Background Art

Being able to control or manipulate certain important biological processes provides numerous benefits to researchers and individuals alike. The ability to effect expression of important receptors, enzymes and activators allows researchers to better understand complex biological mechanisms and create novel and beneficial therapies. For example, H3 acetylation, expression of HMG-CoA reductase and LDL receptor and Sp1 expression in mammals pays a significant role in various health related factors, including but not limited to total and cholesterol levels, cancer prevention, and UV related skin damage. Accordingly, manipulation and control of these biological mechanisms or factors would provide numerous health related benefits and provide researches with new avenues to develop new therapies. Unfortunately, presently there are no known effective methods of safely inhibiting H3 acetylation, reducing expression of HMG-CoA reductase and increasing LDL receptor and Sp1 expression in a mammal. The ability to influence these and other biological factors would be very beneficial to the fields of science and medicine. Accordingly, there exists a need for improved methods of inhibiting H3 acetylation, reducing expression of HMG-CoA reductase and increasing LDL receptor and Sp1 expression in a mammal. The present invention provides these and other related benefits.

As used herein, “biological activity” and “bioactivity” refer to the in vivo activities of a compound or physiological responses that result upon in vivo administration of a compound, composition, or other mixture. Biological activity, thus, encompasses therapeutic effects and pharmaceutical activity of such compounds, compositions and mixtures. Biological activities may be observed and measured in in vitro systems designed to test or use such activities also.

As used herein, the term “biologically active” refers to a molecule having structural, regulatory and or biochemical functions of a naturally occurring lunasin molecule.

As used herein, a “combination” refers to any association between two or among more items.

As used herein, the terms “disease” and “disorder” are used interchangeably to describe a state, signs, and/or symptoms that are associated with any impairment of the normal state of a living animal or of any of its organs or tissues that interrupts or modifies the performance of normal functions, and may be a response to environmental factors (such as malnutrition, industrial hazards, or climate), to specific infective agents (such as worms, bacteria, or viruses), to inherent defect of the organism (such as various genetic anomalies, or to combinations of these and other factors.

As used herein, the term “effective amount” refers to the amount of a composition (e.g., comprising Lunasin) sufficient to effect beneficial or desired results. An effective amount can be administered in one or more administrations, applications or dosages and is not intended to be limited to a particular formulation or administration route.

As used herein, the terms “administration” and “administering” refer to the act of giving a drug, pro-drug, or other agent, or therapeutic treatment (e.g., compositions of the present invention) to a subject (e.g., a subject or in vivo, in vitro, or ex vivo cells, tissues, and organs) and/or to direct, instruct, or advise the use of the composition for any purpose (preferably, for a purpose described herein). Where the administration of one or more of the present compositions is directed, instructed or advised, such direction may be that which instructs and/or informs the user that use of the composition may and/or will provide one or more of the benefits described herein.

Exemplary routes of administration to the human body can be through the eyes (ophthalmic), mouth (oral), skin (topical or transdermal), nose (nasal), lungs (inhalant), oral mucosa (buccal), ear, rectal, vaginal, by injection (e.g., intravenously, subcutaneously, intratumorally, intraperitoneally, etc.) and the like.

Administration which is directed may comprise, for example, oral direction (e.g., through oral instruction from, for example, a physician, health professional, sales professional or organization, and/or radio or television media (i.e., advertisement) or written direction (e.g., through written direction from, for example, a physician or other health professional (e.g., scripts), sales professional or organization (e.g., through, for example, marketing brochures, pamphlets, or other instructive paraphernalia), written media (e.g., internet, electronic mail, or other computer-related media), and/or packaging associated with the composition (e.g., a label present on a package containing the composition). As used herein, “written” includes through words, pictures, symbols, and/or other visible descriptors. Such direction need not utilize the actual words used herein, but rather use of words, pictures, symbols, and the like conveying the same or similar meaning are contemplated within the scope of this invention.

As used herein, the terms “co-administration” and “co-administering” refer to the administration of at least two agent(s) (e.g., composition comprising Lunasin and one or more other agents—e.g., a protease enzyme inhibitor) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the term “treatment” or grammatical equivalents encompasses the prevention, improvement and/or reversal of the symptoms of disease (e.g., skin aging). A composition which prevents or causes an improvement in any parameter associated with disease may thereby be identified as a therapeutic composition. The term “treatment” refers to both therapeutic treatment and prophylactic or preventative measures. For example, those who may benefit from treatment with compositions and methods of the present invention include those already with a disease and/or disorder (e.g., elevated cholesterol levels) as well as those in which a disease and/or disorder is to be prevented (e.g., using a prophylactic treatment of the present invention).

As used herein, the term “at risk for disease” refers to a subject (e.g., a human) that is predisposed to experiencing a particular disease. This predisposition may be genetic (e.g., a particular genetic tendency to experience the disease, such as heritable disorders), or due to other factors (e.g., age, weight, environmental conditions, exposures to detrimental compounds present in the environment, etc.). Thus, it is not intended that the present invention be limited to any particular risk, nor is it intended that the present invention be limited to any particular disease.

As used herein, the terms “individual,” “host,” “subject” and “patient” refer to any animal, including but not limited to, human and non-human animals (for example, without limitation, primates, dogs, cats, cows, horses, sheep, rodents, poultry, fish, crustaceans, etc.) that is studied, analyzed, tested, diagnosed or treated. As used herein, the terms “individual,” “host,” “subject” and “patient” are used interchangeably, unless indicated otherwise.

As used herein, the term “antibody” (or “antibodies”) refers to any immunoglobulin that binds specifically to an antigenic determinant, and specifically binds to proteins identical or structurally related to the antigenic determinant that stimulated their production. Thus, antibodies can be useful in assays to detect the antigen that stimulated their production. Monoclonal antibodies are derived from a single clone of B lymphocytes (i.e., B cells), and are generally homogeneous in structure and antigen specificity. Polyclonal antibodies originate from many different clones of antibody-producing cells, and thus are heterogenous in their structure and epitope specificity, but all recognize the same antigen. Also, it is intended that the term “antibody” encompass any immunoglobulin (e.g., IgG, IgM, IgA, IgE, IgD, etc.) obtained from any source (e.g., humans, rodents, non-human primates, lagomorphs, caprines, bovines, equines, ovines, etc.).

As used herein, the term “antigen” is used in reference to any substance that is capable of being recognized by an antibody.

As used herein, the terms “Western blot,” “Western immunoblot” “immunoblot” and “Western” refer to the immunological analysis of protein(s), polypeptides or peptides that have been immobilized onto a membrane support. The proteins are first resolved by polyacrylamide gel electrophoresis (i.e., SDS-PAGE) to separate the proteins, followed by transfer of the protein from the gel to a solid support, such as nitrocellulose or a nylon membrane. The immobilized proteins are then exposed to an antibody having reactivity towards an antigen of interest. The binding of the antibody (i.e., the primary antibody) is detected by use of a secondary antibody that specifically binds the primary antibody. The secondary antibody is typically conjugated to an enzyme that permits visualization of the antigen-antibody complex by the production of a colored reaction product or catalyzes a luminescent enzymatic reaction (e.g., the ECL reagent, Amersham).

The term “compound” refers to any chemical entity, pharmaceutical, drug, and the like that can be used to treat or prevent a disease, illness, sickness, or disorder of bodily function. Compounds comprise both known and potential therapeutic compounds. Compounds comprise polypeptides such as those described herein.

As used herein, the term “toxic” refers to any detrimental or harmful effects on a subject, a cell, or a tissue as compared to the same cell or tissue prior to the administration of the toxicant.

As used herein, the term “pharmaceutical composition” refers to the combination of an active agent (e.g., Lunasin) with a carrier, inert or active, making the composition especially suitable for diagnostic or therapeutic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “topically” refers to application of the compositions of the present invention to the surface of the skin and mucosal cells and tissues (e.g., alveolar, buccal, lingual, masticatory, or nasal mucosa, and other tissues and cells that line hollow organs or body cavities).

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintrigrants (e.g., potato starch or sodium starch glycolate), and the like. The compositions also can include stabilizers and preservatives. For examples of carriers, stabilizers and adjuvants. (See e.g., Martin, Remington's Pharmaceutical Sciences, 15th Ed., Mack Publ. Co., Easton, Pa. (1975), incorporated herein by reference).

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the terms “gene expression” and “expression” refer to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refer to regulation that increases and/or enhances the production of gene expression products (e.g., RNA or protein), while “down-regulation” or “repression” refer to regulation that decrease production. Molecules (e.g., transcription factors) that are involved in up-regulation or down-regulation are often called “activators” and “repressors,” respectively.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

As used herein “amino acid” refers to any of the naturally occurring amino acids having the standard designations listed in Table 1, below. It also refers to those known synthetic amino acids. Unless otherwise indicated, all amino acid sequences listed in this disclosure are listed in the order from the amino terminus to the carboxyl terminus. As used herein, the abbreviations for any protective groups, amino acids and other compounds, are in accord with their common usage, recognized abbreviations, or the IUPAC-IUB Commission on Biochemical Nomenclature, unless otherwise indicated (see Biochemistry 11: 1726 (1972)). As used herein, amino acid residues are represented by the full name thereof, by the three letter code corresponding thereto, or by the one-letter code corresponding thereto, as indicated in the following table:

TABLE 1

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

As used herein, the terms “peptide,” “polypeptide” and “protein” all refer to a primary sequence of amino acids that are joined by covalent “peptide linkages.” In general, a peptide consists of a few amino acids, typically from 2-50 amino acids. The term “polypeptide” encompasses peptides and proteins, wherein the term “protein” typically refers to large polypeptides and the term “peptide” typically refers to short polypeptides. In some embodiments, the peptide, polypeptide or protein is synthetic, while in other embodiments, the peptide, polypeptide or protein is recombinant or naturally occurring. A “synthetic” peptide is a peptide that is produced by artificial means in vitro (i.e., was not produced in vivo). The term “peptide” further includes modified amino acids (whether naturally or non-naturally occurring), such modifications including, but not limited to, phosphorylation, glycosylation, pegylation, lipidization and methylation.

An “isolated peptide” is a peptide which has been substantially separated from components (e.g., DNA, RNA, other proteins and peptides, carbohydrates and lipids) which naturally accompany it in a cell.

As applied to polypeptides, the term “substantial identity” means that two peptide sequences, when optimally aligned, such as by the programs GAP or BESTFIT using default gap weights, share at least 80% sequence identity, preferably at least 90% sequence identity, more preferably at least 95% sequence identity or more (e.g., 99% sequence identity). Preferably, residue positions which are not identical differ by conservative amino acid substitutions.

The phrase “functionally equivalent” means that the variant, analogue or fragment of a polypeptide retains a desired biological activity in common with the lunasin polypeptide. In at least one embodiment of the present invention, the desired biological activity in common with lunasin is biological activity related to the control, stabilization, or reduction in production or existing levels of cholesterol, LDL cholesterol, total cholesterol, or lipids. Preferably, a given quantity of the analogue, variant or fragment is at least 10%, preferably at least 30%, more preferably at least 50, 60, 80, 90, 95 or 99% as effective as an equivalent amount of the naturally occurring lunasin from which the analogue, variant or fragment is derived. Determination of the relative efficacy of the analogue, variant or fragment can readily be carried out by utilizing a prescribed amount of the analogue, variant or fragment in the one or more of the assay methods of the invention and then comparing the ability of the analogue, variant or fragment to naturally occurring lunasin in tests that measure the ability of the sample to inhibit the acetylation of histone H3, or to effect the expression of HMG Co-A reductase, Sp1 or LDL-receptor.

The term “analogue” as used herein with reference to a polypeptide means a polypeptide which is a derivative of the polypeptide of the invention, which derivative comprises addition, deletion, and/or substitution of one or more amino acids, such that the polypeptide retains substantially the same function as the lunasin polypeptide identified below.

The term “fragment” refers to a polypeptide molecule that is a constituent of the full-length lunasin polypeptide and possesses qualitative biological activity in common with the full-length lunasin polypeptide. The fragment may be derived from the full-length lunasin polypeptide or alternatively may be synthesized by some other means, for example chemical synthesis. By reference to “fragments” it is intended to encompass fragments of a protein that are of at least 5, preferably at least 10, more preferably at least 20 and most preferably at least 30, 40 or 50 amino acids in length and which are functionally equivalent to the protein of which they are a fragment.

The term “variant” as used herein refers to a polypeptide which is produced from a nucleic acid encoding lunasin, but differs from the wild type lunasin in that it is processed differently such that it has an altered amino acid sequence. For example a variant may be produced by an alternative splicing pattern of the primary RNA transcript to that which produces wild type lunasin.

Analogues and variants are intended to encompass proteins having amino acid sequence differing from the protein from which they are derived by virtue of the addition, deletion or substitution of one or more amino acids to result in an amino acid sequence that is preferably at least 60%, more preferably at least 80%, particularly preferably at least 85, 90, 95, 98, 99 or 99.9% identical to the amino acid sequence of the original protein. The analogues or variants specifically include polymorphic variants and interspecies analogues. The analogues and variants of the present invention further may have “conservative” changes, wherein a substituted amino acid has similar structural or chemical properties. One type of conservative amino acid substitution refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. More rarely, a variant may have “non-conservative” changes (e.g., replacement of a glycine with a tryptophan). Similar minor variations may also include amino acid deletions or insertions (i.e., additions), or both. Guidance in determining which and how many amino acid residues may be substituted, inserted or deleted without abolishing biological activity may be found using computer programs well known in the art, for example, DNAStar software. Variants can be tested in functional assays such as those described in the Examples section below.

The term “conservative amino acid substitution” as used herein refers to a substitution or replacement of one amino acid for another amino acid with similar properties within a polypeptide chain (primary sequence of a protein). For example, the substitution of the charged amino acid glutamic acid (Glu) for the similarly charged amino acid aspartic acid (Asp) would be a conservative amino acid substitution.

As used herein “lunasin” refers to the natural, synthetically or recombinantly obtained soybean lunasin polypeptide set forth in (SEQ. ID. 2). Guidance can be found for the identification and screening of functionally equivalent fragments and analogues of Lunasin peptide in the following references: U.S. Pat. No. 6,107287, U.S. Pat. No. 6,544,956, US Patent Application 2003/0229038, filed Nov. 22, 2002, U.S. Pat. No. 6,391,848, U.S. Patent Application No. 10/252,256, filed Sep. 23, 2002, International Application WO 01/72784, filed Mar. 23, 2001, and U.S. patent application Ser. No. 10/302,633, filed Nov. 22, 2002, all of which are hereby incorporated by reference herein in their entirety for all purposes. These disclosures will guide one skilled in the art in identifying functionally equivalent and biologically active fragments, variants and analogues of lunasin.

As used herein “lunasin enriched” refers to compositions containing biologically active levels of naturally occurring lunasin, or a naturally occurring analogue of lunasin, that is at a concentration greater than that at which lunasin is found in the material used as the source of that lunasin or analogue. As used herein “lunasin enriched seed extract” refers to compositions containing biologically active levels of naturally occurring lunasin, or a naturally occurring analogue of lunasin, that is at a concentration at least twice than that at which lunasin is naturally found in the source seed. Without limiting the invention to any particular source of the compositions of the present invention, lunasin enriched compositions can be obtained from soybean, wheat, barley, soy isolates, soy concentrates, or other soy derived products, whether or not commercially obtained.

As used herein “lunasin protecting soy flour” refers to soy flour compositions comprising soy flour and an amount of a protease inhibitor sufficient to protect lunasin, or a analogue, variant or fragment thereof, from complete digestion, wherein the compositions do not have levels of anti-nutritional elements that would cause an adverse effect in an individual who ingested them.

As used herein “digested” refers to the treatment of a polypeptide with a digestive material that breaks it down into its component amino acids. Examples of digestive materials that can be used are well known in the art, and include, without limitation, pancreatin and other proteases such as trypsin, chymotrypsin, pepsin, Proteinase K, thermolysin, thrombin, Arg-C proteinase, Asp-N endopeptidase, AspN endopeptidase+N-terminal Glu, BNPS-Skatole, CNBr, clostripain, formic acid, glutamyl endopeptidase, iodosobenzoic acid, LysC, LysN, NTCB (2-nitro-5-thiocyanobenzoic acid), and Staphylococcal peptidase.

As used herein “partially digested biologically active” in relation to a polypeptide refers to the treatment of a polypeptide with a digestive material under conditions that increase the biological activity of the polypeptide.

The phrase “combination therapy” embraces the administration of a composition of the present invention in conjunction with another pharmaceutical agent that is indicated for treating or preventing a disorder, as part of a specific treatment regimen intended to provide a beneficial effect from the co-action of these therapeutic agents.

Referenced herein are trade names for components including various ingredients utilized in the present invention. The inventors herein do not intend to be limited by materials under a certain trade name. Equivalent materials (e.g., those obtained from a different source under a different name or reference number) to those referenced by trade name may be substituted and utilized in the descriptions herein.

The compositions herein may comprise, consist essentially of, or consist of any of the elements as described herein.

The practice of the present invention will employ, unless otherwise indicated, conventional techniques of molecular biology (including recombinant techniques), microbiology, cell biology, biochemistry, immunology, and protein kinetics, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); and Mass isotopomer distribution analysis at eight years: theoretical, analytic and experimental considerations by Hellerstein and Neese (Am J Physiol 276 (Endocrinol Metab. 39) E1146-E1162, 1999), all of which are incorporated herein by reference in their entirety. Furthermore, procedures employing commercially available assay kits and reagents will typically be used according to manufacturer-defined protocols unless otherwise noted.

### SUMMARY OF THE INVENTION

The present invention relates generally to a class of peptides that provide mammals with a variety of health related benefits. More specifically, the present invention involves using soy peptides to inhibit H3 acetylation, reduce expression of HMG-CoA reductase and increase LDL receptor and Sp1 expression in a mammal, protect against, prevent, or reduce: 1) the expression of Matrix metalloproteinase (MMP-1), 2) collagen breakdown, 3) photoaging and 4) the formation of skin wrinkles.

In at least one exemplary embodiment of the present invention, a method of inhibiting PCAF from acetylating H3 in a mammal is provided. The method includes providing an effective amount of lunasin peptides to a mammal to inhibit H3 acetylation in the mammal.

In at least one other exemplary embodiment of the present invention, a method of reducing expression of HMG-CoA reductase in a mammal is provided. The method includes providing an effective amount of lunasin peptides to a mammal to reduce expression of HMG-CoA reductase in the mammal.

In at least one other exemplary embodiment of the present invention, a method of increasing LDL receptor expression in a mammal is provided. The method includes providing an effective amount of lunasin peptides to a mammal to increase LDL receptor expression in the mammal.

In at least one other exemplary embodiment of the present invention, a method of increasing Sp1 transcriptional activator expression in a mammal is provided. The method includes providing an effective amount of lunasin peptides to a mammal to increase Sp1 transcriptional activator expression in the mammal.

In one aspect of at least one embodiment of the present invention, the effective amount of lunasin peptides that inhibit H3 acetylation, reduce expression of HMG-CoA reductase, increase LDL receptor expression or increases Sp1 transcriptional activator expression in a mammal is 25 to 100 mg daily.

In another aspect of at least one embodiment of the present invention, the lunasin peptides include lunasin peptides or lunasin peptide derivatives.

In yet another aspect of at least one embodiment of the present invention, the lunasin peptides are obtained from, soy, seed bearing plants other than soy, using recombinant DNA techniques and synthetic polypeptide production or any combination thereof.

In yet another aspect of at least one embodiment of the present invention, the method includes providing an effective amount of one or more protease enzyme inhibitors to the lunasin peptides.

In at least one exemplary embodiment of the present invention, a method for protecting against photoaging of skin in an individual, is provided, comprising: (a) providing: (i) an individual desiring to prevent photoaging of skin and, (ii) a composition comprising a compound selected from the group consisting of the peptide of SEQ ID NO 2 and a functionally equivalent variant, fragment or analogue of said peptide; and (b) administering said composition to said subject to protect against photoaging.

In one aspect of at least one embodiment of the present invention, said compound is obtained from soybean, wheat or barley. In another aspect, the compound is obtained by producing, extracting and purifying said compound using recombinant DNA techniques. In yet another aspect, said compound is obtained by synthetic polypeptide production. In yet another aspect of at least one embodiment of the present invention, said individual is a human. In yet another aspect of the present invention, administering comprises topical administration of the composition. In yet another aspect of the present invention the composition is in the form of a semi-solid formulation, liquid, gel, suspension, or aerosol spray. And in yet another aspect of the invention, said composition further comprises chymotrypsin inhibitor. In still further aspects of the present invention, said compound is administered to said individual at between 5 μg/ml and 50 μg/ml.

In at least one exemplary embodiment of the present invention, a method for protecting against collagen breakdown in the skin in an individual is provided, comprising: (a) providing: (i) an individual desiring to prevent collagen breakdown in the skin and, (ii) a composition comprising a compound selected from the group consisting of the peptide of SEQ ID NO 2 and a functionally equivalent variant, fragment or analogue of said peptide; and (b) administering said composition to said subject to protect against collagen breakdown.

In one aspect of at least one embodiment of the present invention, said compound is obtained from soybean, wheat or barley. In another aspect, the compound is obtained by producing, extracting and purifying said compound using recombinant DNA techniques. In yet another aspect, said compound is obtained by synthetic polypeptide production. In yet another aspect of at least one embodiment of the present invention, said individual is a human. In yet another aspect of the present invention, administering comprises topical administration of the composition. In yet another aspect of the present invention the composition is in the form of a semi-solid formulation, liquid, gel, suspension, or aerosol spray. And in yet another aspect of the invention, said composition further comprises chymotrypsin inhibitor. In still further aspects of the present invention, said compound is administered to said individual at between 5 μg/ml and 50 μg/ml.

In at least one exemplary embodiment of the present invention, a method for protecting against wrinkling of the skin in an individual is provided, comprising: (a) providing: (i) an individual desiring to prevent wrinkling of skin and, (ii) a composition comprising a compound selected from the group consisting of the peptide of SEQ ID NO 2 and a functionally equivalent variant, fragment or analogue of said peptide; and (b) administering said composition to said subject to protect against wrinkling of the skin.

In one aspect of at least one embodiment of the present invention, said compound is obtained from soybean, wheat or barley. In another aspect, the compound is obtained by producing, extracting and purifying said compound using recombinant DNA techniques. In yet another aspect, said compound is obtained by synthetic polypeptide production. In yet another aspect of at least one embodiment of the present invention, said individual is a human. In yet another aspect of the present invention, administering comprises topical administration of the composition. In yet another aspect of the present invention the composition is in the form of a semi-solid formulation, liquid, gel, suspension, or aerosol spray. And in yet another aspect of the invention, said composition further comprises chymotrypsin inhibitor. In still further aspects of the present invention, said compound is administered to said individual at between 5 μg/ml and 50 μg/ml.

### BRIEF DESCRIPTION OF DRAWINGS

The above-mentioned features and objects of the present disclosure will become more apparent with reference to the following description taken in conjunction with the accompanying drawings wherein like reference numerals denote like elements and in which:

FIG. 1 shows the 2S albumin protein encoded by Gm2S 1 cDNA (SEQ ID NO 1). Arrows indicate endoproteolytic sites that give rise to small subunit (“lunasin”) (SEQ ID NO 2) and the large subunit (methionine rich protein). Important regions in both subunits are indicated.

FIG. 2 is a photograph of a Western blot analysis (top) and a table (below) showing densitometer values indicating the relative levels of expression of HMG-CoA reductase in HepG2 cells that were (CFM+LS (24)) or were not (CFM) treated with lunasin for 24 hours prior to incubation in cholesterol free media (CFM) for 24 hours to activate sterol regulatory element binding proteins (SREBP.) After incubations, total protein was extracted and 10 ug protein was loaded onto 10% Tris-glycine gels, electroblotted onto nitrocellulose membrane, and immunostained with primary antibodies raised against HMG-CoA reductase and actin (to show equal loading of proteins.) Spot densitometer values represent mean and standard deviation of data from three separate experiments.

FIG. 3 is a photograph of a Western blot analysis (top) and a table (below) showing densitometer values indicating the relative levels of expression of LDL receptor in HepG2 cells that were (CFM +LS(24)) or were not (CFM) treated with lunasin for 24 hours prior to incubation in cholesterol free media (CFM) for 24 hours to activate SREBP. After incubations, total protein was extracted and 10 ug protein was loaded onto 10% Tris-glycine gels, electroblotted onto nitrocellulose membrane, and immunostained with primary antibodies raised against LDL-receptor and actin (to show equal loading of proteins.) Spot densitometer values represent mean and standard deviation of data from three separate experiments.

FIG. 4 is a photograph of a Western blot analysis (top) and a table (below) showing densitometer values indicating the relative levels of expression of Sp1 in HepG2 cells that were grown from confluence in growth media for 24 hours before growth media was replaced with fresh growth media (Media), media with lunasin (Media+LS) or cholesterol free media with lunasin (CFM+LS) or without lunasin (CFM). Samples were then incubated for 24 or 48 hours as indicated. After incubations, total protein was extracted and 10 ug protein was loaded onto 10% Tris-glycine gels, electroblotted onto nitrocellulose membrane, and immunostained with primary antibodies raised against Sp1 and actin (to show equal loading of proteins.) Spot densitometer values represent data from one experiment.

FIG. 5 shows the western blots from experiments on PCAF reaction products demonstrating that lunasin caused a dramatic reduction in histone H3 acetylation. Acid extracted protein from untreated (untrt) HeLa cells was used as template in histone acetylase reactions using HAT enzyme, PCAF, in the presence or absence of 10 uM lunasin. Reaction products were immunoblotted and stained with antibodies against diacetylated histone H3. Untrt (−) is the histone template control, NaB (+) correspond to acid extracted histones from NaButyrate treated HeLa cells (positive control). Boxed signal indicates significant decrease in H3 acetylation upon addition of 10 uM lunasin compared with no lunasin application. Numbers in parenthesis indicate densitometer readings relative to the untreated control (set as 1) in PCAF HAT reaction products.

FIG. 6 shows the western blots from experiments on PCAF HAT reaction products demonstrating that lunasin caused a dramatic reduction in histone H3 acetylation. Acid extracted histones isolated from untreated (untrt) HeLa cells were used in PCAF HAT reactions, immunoblotted and stained with antibodies to H3 Ac-Lys9 and H3 Ac-Lys14. Untrt (−) is the histone template control, NaB (+) correspond to acid extracted histones from NaButyrate treated HeLa cells (posititve control), +Lun correspond to 10 uM lunasin treated histone template and −Lun correspond to non-lunasin treated. Boxed signal indicate decreased H3 Lys 14 acetylation by PCAF acetylase enzyme in the presence of lunasin. Numbers in parenthesis indicate densitometer readings relative to the signal level of lunasin/lunasin treatment (set as 1) in immunoblots stained with Ac-Lys14 H3.

### DETAILED DESCRIPTION

Lunasin is a recently discovered bioactive component in soy with a novel chromatin-binding property and epigenetic effects on gene expression (1, 2). The lunasin soy peptide is heat stable, water soluble and found in significant amounts in select soy protein preparations (3). Studies show that it can get inside mammalian epithelial cells through its RGD cell adhesion motif, bind preferentially to deacetylated histones and inhibit histone H3 and H4 acetylation (4). There is growing evidence that cellular transformation, responses to hormones and dietary and environmental effects involve epigenetic changes in gene expression, which are modulated by the reversible processes of DNA methylation-demethylation and histone acetylation-deacetylation (5, 6). Lunasin is the first natural substance to be identified as a histone acetylase inhibitor, although it does not directly affect the histone acetylase enzyme. It inhibits H3 and H4 acetylation by binding to specific deacetylated lysine residues in the N-terminal tail of histones H3 and H4, making them unavailable as substrates for histone acetylation. The elucidation of the mechanism of action makes lunasin an important molecule for research studies to understand the emerging role of epigenetics and chromatin modifications in important biological processes.

The study on the effect of lunasin on prostate carcinogenesis at the University of California at Davis revealed the effects of lunasin on histone H4 modifications and the up regulation of chemopreventive genes, (7). However, until now, the specific effect of lunasin binding to deacetylated H3 N-terminal tail and the inhibition of H3 histone acetylation in biological systems had not yet been investigated. To determine the specific biological effect of lunasin binding to deacetylated histone H3 and inhibition of acetylation, the induction of genes involved in cholesterol biosynthesis by the sterol regulatory element binding proteins (SREBP) was chosen as a biological model. This biological model was chosen because activation of SREBPs by sterol depletion results in the increased acetylation of histone H3 but not histone H4, by the histone acetylase enzyme PCAF, in chromatin proximal to the promoters of HMG-CoA reductase and the LDL receptor genes (8) and SREBP activation results in the increased recruitment of co-regulatory factors, CREB to the promoter of HMG-CoA reductase gene, and Sp1 to the promoter of LDL receptor gene (8).

Our studies on in vitro histone acetylase (HAT) assays show that lunasin significantly inhibits histone H3 acetylation (specifically lysine 14 in H3 N-terminal tail) by the histone acetylase enzyme, PCAF. Cell culture experiments using HepG2 liver cells show that synthetic lunasin can significantly reduce HMG-CoA reductase expression and increase LDL receptor gene expression in cholesterol-free media similar to the effects of statin (cholesterol-lowering) drugs. Our studies have also shown that the increase in LDL receptor expression coincides with the increase in Sp1 expression in cholesterol-free media. Based on these studies, a molecular mechanism of action is proposed wherein synthetic lunasin reduces total and LDL cholesterol levels by binding to deactylated histone H3 and inhibiting histone H3 acetylation by PCAF (through its association with the CREB-binding protein), thereby reducing SREBP activation of the HMG-CoA reductase gene resulting in lower endogenous cholesterol biosynthesis, and by increasing the expression of the Sp1 co-activator in sterol-free media and upon SREBP activation, an increased amount of membrane bound LDL receptors is expressed leading to significant reduction of plasma LDL cholesterol levels (9).

Our data described and shown below demonstrates that lunasin (a.k.a. lunastantin) is the bioactive agent from soy responsible for inhibiting H3 acetylation, reducing expression of HMG-CoA reductase and increasing LDL receptor and Sp1 expression in a mammal.

Our surprising finding that lunasin inhibits H3 acetylation, reduces expression of HMG-CoA reductase and increases LDL receptor and Sp1 expression in a mammal can be used for numerous health related benefits, including but not limited to, to lower total or LDL cholesterol levels or to prevent, control or treat cancers in mammals. These effects of lunasin can be further increased by developing formulations of lunasin and lunasin derivatives that are optimized for adsorption and delivery to the liver.

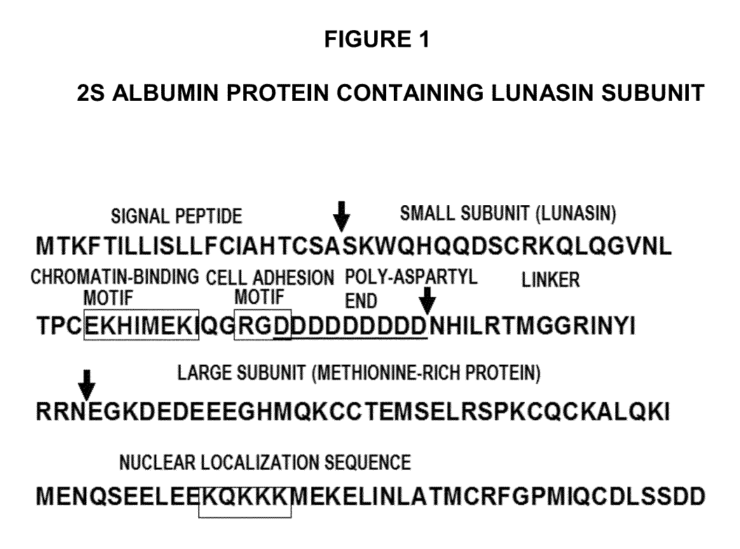

Lunasin is the small subunit peptide of a cotyledon-specific 2S albumin. FIG. 1 shows the 2S albumin protein and the small lunasin subunit. It has been shown that constitutive expression of the lunasin gene in mammalian cells disturbs kinetochore formation and disrupts mitosis, leading to cell death (2). When applied exogenously in mammalian cell culture, the lunasin peptide suppresses transformation of normal cells to cancerous foci that are induced by chemical carcinogens and oncogenes. To elucidate its chemopreventive mechanism of action, we have shown that lunasin (a) is internalized through its RGD cell adhesion motif, (b) colocalizes with hypoacetylated chromatin in telomeres at prometaphase, (c) binds preferentially to deacetylated histone H4, which is facilitated by the presence of a structurally conserved helical motif found in other chromatin-binding proteins, (d) inhibits histone H3 and H4 acetylation, and (e) induces apoptosis in EIA-transfected cells (4). Based on these results, a novel chemopreventive mechanism has been proposed wherein lunasin gets inside the nucleus, binds to deacetylated histones, prevents their acetylation and inhibits gene expression like those controlled by the Rb tumor suppressor and h-ras oncogene.

Lunasin's Effect On Expression Of Sp1 Coactivator

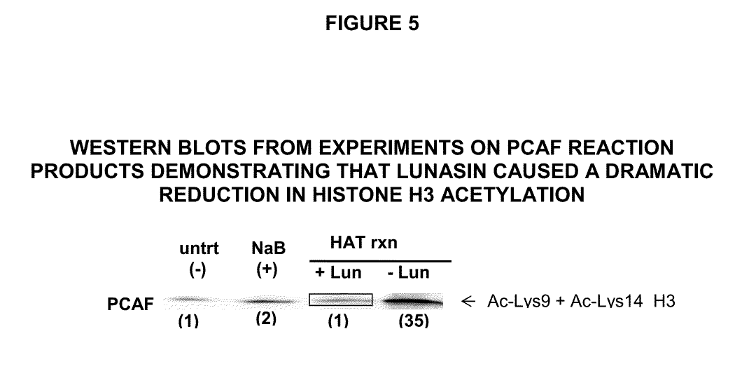

Inhibition of H3 histone acetylation by PCAF histone acetylase enzyme is required for the SREBP activation of genes involved in cholesterol biosynthesis including HMG-CoA reductase (9). Previous study has shown that lunasin is a potent inhibitor of histone H3 acetylation in mammalian cells exposed to the histone deacetylase inhibitor, sodium butyrate (NaB) (4). To determine the effect of lunasin on histone H3 acetylation by PCAF, HAT assay reaction using acid-extracted histones from untreated HeLa cells as template was conducted. Immunoblotted reaction products have been stained with antibodies against diacetylated histone H3 (Ac-Lys9+Ac-Lys14) and the details of our experiment and its results are shown and described in FIG. 5. In brief, the HAT enzyme, PCAF, is shown to increase significantly histone H3 acetylation in the absence of lunasin (35-fold increase). However, the addition of lunasin in the PCAF reaction, resulted in dramatic reduction of histone H3 acetylation, indicating that lunasin is a potent inhibitor of histone H3 acetylation catalyzed by the PCAF acetylase enzyme.

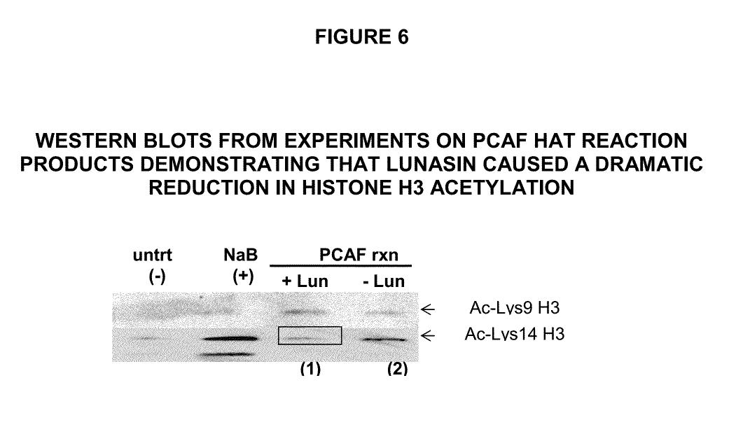

To determine the specific lysine residue in histone H3 that is inhibited by lunasin from being acetylated, immunoblotted products of PCAF acetylase reactions were hybridized with antibodies raised against acetylated Lys 9 and acetylated Lys 14 in H3 terminal tails. The results and details of our experiments, as shown and described in FIG. 6, demonstrate that lunasin specifically binds to Lys 14, preventing it from being acetylated by PCAF.

In one exemplary embodiment of the present invention, a method of inhibiting H3 acetylation in a mammal is provided. The method includes providing an effective amount of lunasin peptides to a mammal to inhibit H3 acetylation in the mammal.

In another exemplary embodiment of the present invention, a method of reducing expression of HMG-CoA reductase in a mammal is provided. The method includes providing an effective amount of lunasin peptides to a mammal to reduce expression of HMG-CoA reductase in the mammal.

In yet another exemplary embodiment of the present invention, a method of increasing LDL receptor expression in a mammal is provided. The method includes providing an effective amount of lunasin peptides to a mammal to increase LDL receptor expression in the mammal.

In yet another exemplary embodiment of the present invention, a method of increasing Sp1 transcriptional activator expression in a mammal is provided. The method includes providing an effective amount of lunasin peptides to a mammal to increase Sp1 transcriptional activator expression in the mammal.

In one aspect of at least one embodiment of the present invention, the effective amount of lunasin peptides that inhibit H3 acetylation, reduce expression of HMG-CoA reductase, increase LDL receptor expression or increases Sp1 transcriptional activator expression in a mammal is 25 to 100 mgs daily. It should be appreciated that the effective amount of lunasin will depend, at least in part, on the size, weight, health and desired goals of the mammals consuming the compositions. Accordingly, it is believed that in at least one embodiment, the effective amount of lunasin provided to the mammal is 25 mg to 100 mg daily.

In another aspect of at least one embodiment of the present invention, the lunasin peptides include lunasin peptides or lunasin peptide derivatives. It should also be appreciated that the present invention includes the use of lunasin peptide derivatives, which are any peptides that contain the same functional units as lunasin. It should also be appreciated the products and compositions of the present invention can be used in, foods, powders, bars, capsules, shakes and other well known products consumed by mammals or used separately.

In yet another aspect of at least one embodiment of the present invention, the lunasin peptides are obtained from, soy, seed bearing plants other than soy, using recombinant DNA techniques and synthetic polypeptide production or any combination thereof.

In yet another aspect of at least one embodiment of the present invention, the method includes providing an effective amount of one or more protease enzyme inhibitors with or without the lunasin peptides. The protease enzyme inhibitors act to protect lunasin from digestion and facilitate absorption and delivery to the appropriate target areas. Examples of appropriate protease enzyme inhibitors include, but are not limited to, pancreatin, trypsin and/or chymotrypsin inhibitors. It should be appreciated that the scope of the present inventions includes the use of the lunasin and/or lunasin derivatives with any other composition or product that is known or believed to facilitate lunasin's absorption or delivery in a mammal.

Photoprotective Effects.

The activity of lunasin peptides in prevention of histone H3 acetylation has additional applications for the protection against and prevention of the expression of

Matrix metalloproteinase (MMP-1), collagen breakdown, photoaging and the formation of skin wrinkles.

The present invention encompasses the use of lunasin peptides for the protection against and prevention of the expression of Matrix metalloproteinase (MMP-1), collagen breakdown in the skin, photoaging of the skin, premature aging of the skin and the formation of skin wrinkles.

It has been previously shown that UV irradiation stimulates histone H3 acetylation at Lys14 (19). Further, an increase in histone H3 acetylation results in chromatin modification of gene promoters and is associated with increased accessibility of chromatin by transcriptional factors involved in the activation and expression of repressed genes (19). The histone acetylase (HAT) enzyme that specifically acetylates H3-Lys14 is PCAF (p300/CBP-associated factor) is the mammalian homologue of the yeast HAT, Gcn5p that was evaluated in the studies referenced.

Matrix metalloproteinase (MMP-1), commonly known as interstitial collagenase, is known to degrade collagen and extracellular matrix components (20).

Its expression promotes the breakdown of collagen and connective tissues, and excessive damage to connective tissue is associated with dermal photoaging (20). MMP-1 triggers long term detrimental effects such as premature aging. UV radiation from the sun induces the expression of MMP-1 by increasing histone H3 acetylation (20). Inhibition of histone H3 acetylation by HAT inhibitors reduces UV-induced expression of MMP-1 (20). It is thus desirable to interfere with the acetylation of histone H3 in order to reduce the expression of MMP-1 and as a result reduce collagen breakdown and the photoaging and the premature aging associated with it.

As shown herein, the binding of lunasin to H3 masks deacetylated H3-Lys14 from acetylation, and, as a result lunasin inhibits H3 acetylation by PCAF.

Without being limited to any particular mechanism of action, it is believed that by binding to chromatin, specifically to the N-terminal tail of deacetylated histone H3, lunasin will prevent the UV-induced PCAF acetylation of H3-Lys14 in the chromatin near the MMP-1 promoter, thereby interfering with the expression of MMP-1 and preventing the damage to collagen, photoaging, premature skin aging and skin wrinkling. The inhibition of H3 acetylation in the epidermal layer of skin cells will interfere with the expression of MMP-1 that would otherwise be activated by exposure to UV radiation from the sun. The interference with MMP-1 expression as a result of lunasin treatment will lead to a decrease in the expression of MMP-1, will protect against collagen breakdown in the skin and result in protection against the effects of photoaging and premature aging of the skin and protection against skin wrinkling that are the result of sun exposure.

Administration

The compositions can be administered using a number of different routes including oral administration, topical administration, transdermal administration, or injection directly into the body. Administration of compositions for use in the practice of the present invention can be systemic (i.e., administered to the subject as a whole via any of the above routes) or localized (i.e., administered to the specific location of the particular disease or pathological condition of the subject via any of the above routes). In a preferred embodiment of the present invention, the compositions to decrease the expression of Matrix metalloproteinase (MMP-1), protect against collagen breakdown in the skin, protect against skin photoaging, or protect against skin wrinkling in an individual are administered by topical administration.

The present methods, kits, and compositions can also be used in “combination therapy” with another composition or treatment that is indicated for treating or preventing a disorder.

Dosing

In one exemplary embodiment of the present invention, a product containing an effective amount of lunasin peptides to prevent the expression of Matrix metalloproteinase (MMP-1), reduce collagen breakdown, or prevent dermal photoaging in an individual that is treated with the product is provided.

Depending upon the particular needs of the individual subject involved, the compositions of the present invention can be administered in various doses to provide effective treatment concentrations based upon the teachings of the present invention. Factors such as the activity of the selected compositions, the physiological characteristics of the subject, the extent or nature of the subject's disease or pathological condition, and the method of administration will determine what constitutes an effective amount of the selected compositions. Generally, initial doses will be modified to determine the optimum dosage for treatment of the particular subject. Suitable dosages can be chosen by taking into account any or all of such factors as the size, weight, health, age, and sex of the human or individual, the desired goals of the patient, the severity of the pathological condition for which the composition is being administered, the response to treatment, the type and quantity of other medications being given to the patient that might interact with the composition, either potentiating it or inhibiting it, and other pharmacokinetic considerations such as liver and kidney function. These considerations are well known in the art and are described in standard textbooks.

A therapeutically effective amount of any embodiment of the present invention is determined using methods known to pharmacologists and clinicians having ordinary skill in the art. Blood levels of the composition can be determined using routine biological and chemical assays and these blood levels can be matched to the route of administration. The blood level and route of administration giving the most desirable level of cholesterol reduction can then be used to establish an “effective amount” of the pharmaceutical composition for treatment.

This same method of titrating a composition in parallel with administration route can be used to ascertain a therapeutically effective amount of the compositions of the present invention for treating any and all disorders described herein. In addition, animal models as described below can be used to determine applicable dosages to treat or prevent a particular disease or pathological condition. Typically, dosage-effect relationships from in vitro or in vivo tests initially can provide useful guidance on the proper doses for subject administration.

In one embodiment of the present invention related to decreasing the expression of Matrix metalloproteinase (MMP-1), protecting against collagen breakdown in the skin, protecting against skin photoaging, or protecting against skin wrinkling in an individual, methods and compositions of the invention encompass a dose of a composition comprising lunasin, or a functionally equivalent variant, analogue or fragment of lunasin, of about 5 ng to about 1000 g, or about 100 ng to about 600 mg, or about 1 μg to about 500 μg, or about 5 μg/ml and 50 μg/ml. Illustratively, a dosage unit of a composition of the present invention can typically contain, for example, without limitation, about 5 ng, 50 ng 100 ng, 500 ng, 1 μg, 10 μg, 100 μg, 250 μg, 500 μg, 1 mg, 10 mg, 20 mg, 40 mg, 80 mg, 100 mg, 125 mg, 150 mg, 200 mg, 250 mg, 300 mg, 350 mg, 400 mg, 450 mg, 500 mg, 550 mg, 600 mg, 700 mg, 800 mg, 900 mg, 1g, 5g, 10 g, 20 g, 30 g, or 40 g of a composition of the present invention. In certain preferred embodiments of the present invention, compositions of the present invention contain about 1 to 500 μg, preferably 5 μg/ml and 50 μg/ml, more preferably approximately 250 μg per dosage of lunasin, or fragments, variants and analogues of lunasin.

Exemplary dosages for lunasin, or fragments, variants and analogues thereof, in accordance with the teachings of the present invention, range from 0.1 μg to 200 mg, preferably, 1 μg to 100 mg, more preferably 25 μg to 500 μg for humans and other individuals, although alternative dosages are contemplated as being within the scope of the present invention.

In certain preferred embodiments of the present invention for compositions and methods for topical administration, lunasin, or fragments, variants and analogues thereof, is present at a level of between 25 μg/ml and 25 mg/ml, more preferably between 50 μg/ml and 1 mg/ml, more preferably between 100 μg/ml and 500 μg/ml, even more preferably, approximately 250 μg/ml.

Existing literature offers additional guidance in determining appropriate dosage for topical administration of lunasin or fragments, variants and analogues thereof for applications of the present invention. Guidance on a lunasin effective dosage range can be obtained, for example, from Table 1 of reference (4) below (Galvez, A F, et al., Cancer Res. 61:7473-7478 (2001)), wherein dosages of 10 nM to 10,000 nM of lunasin were evaluated to determine lunasin efficacy in reducing tumor formation in mammalian cells. Lunasin dosages of 1,000 nM (equivalent to 5 μg/ml) to 10,000 nM (equivalent to 50 μg/ml) all showed statistically significant tumor reduction activity compared to the control group, but were not statistically significantly different in their ability to reduce tumor formation induced by chemical carcinogens. The chemopreventive effect of lunasin is linked to the ability of lunasin to bind to deacetylated histones and inhibit histone acetylation (4). This and other dosage guidance in the literature will be helpful in determining appropriate dosage for various topical applications including those related to decreasing the expression of Matrix metalloproteinase (MMP-1), protecting against collagen breakdown in the skin, protecting against skin photoaging, or protecting against skin wrinkling in an individual.

In certain preferred embodiments of the present invention for compositions and methods for oral administration, lunasin, or fragments, variants and analogues thereof, is provided to an individual at a level of between 0.01 mg/Kg and 100 mg/Kg body weight of an individual, preferably 0.05 mg/Kg and 50 mg/Kg, more preferably between 0.5 mg/Kg and 2.5 mg/Kg, and even more preferably between 0.2 mg/Kg and 1.5 mg/Kg.

A dose can be administered in one to about four doses per day, or in as many doses per day to elicit a therapeutic effect. The dosage form can be selected to accommodate the desired frequency of administration used to achieve the specified dosage, as well as the route of delivery.

The amount of therapeutic agent necessary to elicit a therapeutic effect can be experimentally determined based on, for example, the absorption rate of the agent into the blood serum, or the dermal layer of the skin for topical applications, and the bioavailability of the agent. Determination of these parameters is well within the skill of the art.

Formulations.

The invention also concerns formulations containing the compositions of the present invention. The products and compositions of the present invention can be used alone or in foods, powders, bars, capsules, shakes and other well known products consumed by individuals.

In one preferred embodiment the compositions of the present invention are together with a dietary suitable excipient, diluent, carrier, or with a food. In a preferred embodiment of the present invention, the formulation is in the form of a pill, tablet, capsule, powder, food bar or similar dosage form.

The formulations may be a variety of kinds, such as nutritional supplements, pharmaceutical preparations, vitamin supplements, food additives or foods supplemented with the specified compositions of the invention, liquid or solid preparations, including drinks, sterile injectable solutions, tablets, coated tablets, capsules, powders, drops, suspensions, or syrups, ointments, lotions, creams pastes, gels, or the like.

The formulations may be packaged in convenient dosage forms, and may also include other active ingredients, and/or may contain conventional excipients, pharmaceutically acceptable carriers and diluents. The inclusion of the compositions of the present invention in herbal remedies and treatments is also a preferred part of the invention.

Preferred formulations for topical applications of the compositions of the present invention for both pharmaceutical and cosmetic use will employ excipients that are suitable for topical application. Topical formulations typically are gels, salves, powders, or liquids, though controlled formulations which release defined amounts of active ingredient at the desired surface are also desirable. The formulations may contain materials which enhance the permeability of the active moieties through the epidermis. Such penetrants include, for example, DMSO, various bile salts, non-toxic surfactants and the like. Standard ingredients for cosmetic/pharmaceutical compositions are well known in the art; formulations for topical application of pharmaceuticals are found in Remington's Pharmaceutical Sciences, latest edition, Mack Publishing Co., Easton, Pa., incorporated herein by reference. Cosmetic formulations are widely varied and well known to practitioners.

While the products, compositions and related methods have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims. All the patents, journal articles and other documents discussed or cited above are herein incorporated by reference.

### EXAMPLES

The following examples are provided in order to demonstrate and further illustrate certain preferred embodiments and aspects of the present invention and are not to be construed as limiting the scope thereof.

### Example 1

Lunasin Reduces Expression of HMG-CoA Reductase, Increases Expression of LDL Receptor.

The lowering of serum cholesterol by statin drugs is achieved by competitively inhibiting the HMG-CoA reductase, the rate limiting enzyme in the body's metabolic pathway for synthesis of cholesterol. By reducing endogenous cholesterol synthesis, statins also cause liver cells to up regulate expression of the LDL receptor, leading to increased clearance of low-density lipoprotein (LDL) from the bloodstream (9). In 1985, Michael Brown and Joseph Goldstein received the Nobel Prize in Medicine for their work in clarifying this LDL-lowering mechanism.

Transcriptional regulation of HMG-CoA reductase and LDL receptor is controlled by the Sterol Regulatory Element-Binding Protein-1 and -2 (SREBP). This protein binds to the sterol regulatory element (SRE) located on the 5′ end of the reductase and the LDL receptor genes. When SREBP is inactive, it is bound to the ER or nuclear membrane. When cholesterol levels fall, SREBP is released from the membrane by proteolysis and migrates to the nucleus, where it binds to the SRE to up regulate transcription of HMG-CoA reductase and LDL receptor (8, 9).

In cell culture of HepG2 liver cells, it is possible to activate SREBP and increase the expression of HMG-CoA reductase and LDL-receptor by removing cholesterol in the growth media. This can be achieved by exposing the cells to serum-free media for 24 hours (15, 16).

The following related experiments were performed to evaluate the effect of lunasin on HMG-CoA reductase expression and LDL-receptor expression.

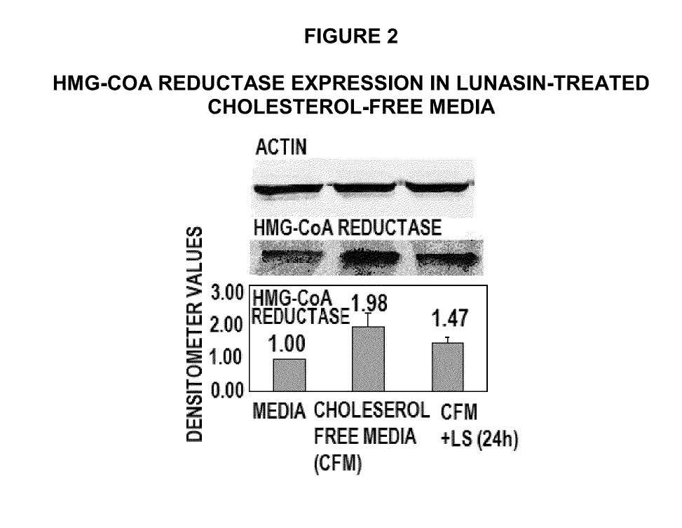

In the first experiment, HepG2 cells (1×10⁶) were treated with or without 10 uM synthetic lunasin in DMEM with 10% FBS for 24 hours before growth media was replaced with cholesterol-free media to activate SREBP. After 24 hours, total protein was extracted and 10 ug protein was loaded onto 10% Tris-glycine gels, electroblotted onto nitrocellulose membrane, and immunostained with primary antibodies raised against HMG-CoA reductase and actin (to show equal loading of proteins). Spot densitometer values are obtained by digital scanning and Un-Scan It software, and represent mean and standard deviation of data from three separate experiments. The results are shown in FIG. 2.

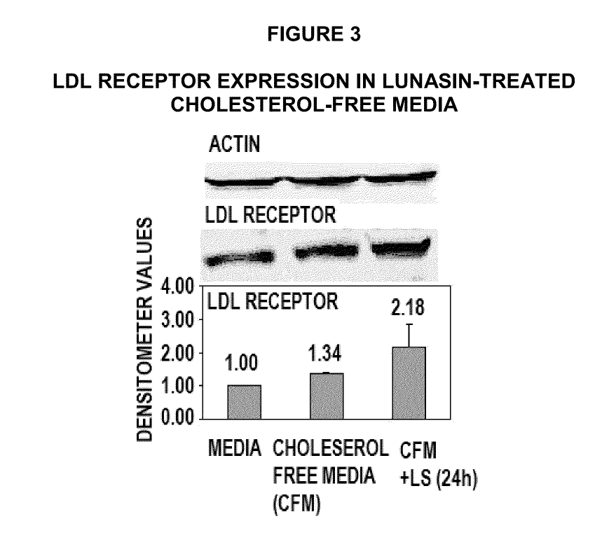

In the second experiment, HepG2 cells (1×10⁶) were treated with or without 10 uM synthetic lunasin in DMEM with 10% FBS for 24 hours before growth media is replaced with cholesterol-free media to activate SREBP. After 24 hours, total protein was extracted and 10 ug proteins loaded onto 10% Tris-glycine gels, electroblotted onto nitrocellulose membrane, and immunostained with primary antibodies raised against LDL-receptor and actin (to show equal loading of proteins). Spot densitometer values were obtained by digital scanning and Un-Scan It software, and represent mean and standard deviation of data from three separate experiments. The results are shown in FIG. 3.

FIGS. 2 and 3 show the upregulation of HMG-CoA reductase (98% increase) and LDL-receptor (34% increase) when HepG2 cells are grown in cholesterol-free media for 24 hours. However when lunasin is added to the cholesterol-free media, the expression of the HMG-CoA reductase is reduced by more than 50% (FIG. 2), while the expression of LDL-receptor has increased by more than 60% (FIG. 3).

This effect of lunasin is similar to statin drugs that reduces endogenous cholesterol synthesis by inhibiting HMG-CoA reductase activity, which leads to increased LDL receptor expression. However, while it is not intended that the present invention be limited to any precise mechanism or mode of action, the mode of action of lunasin is believed to differ from statin drugs in that it appears to inhibit expression of HMG-CoA reductase at the transcriptional level, rather than on inhibiting its enzyme activity. Like statin drugs, lunasin up regulates the expression of LDL-receptor gene. Again, while it is not intended that the present invention be limited to any precise mechanism or mode of action, the contrasting effect of lunasin on these two SREBP-controlled genes can be explained by the selective recruitment of different co-regulatory transcription factors to two separate cholesterol-regulated promoter/regulatory sequences.

### Example 2

Lunasin's Effect on Expression of Sp1 Coactivator

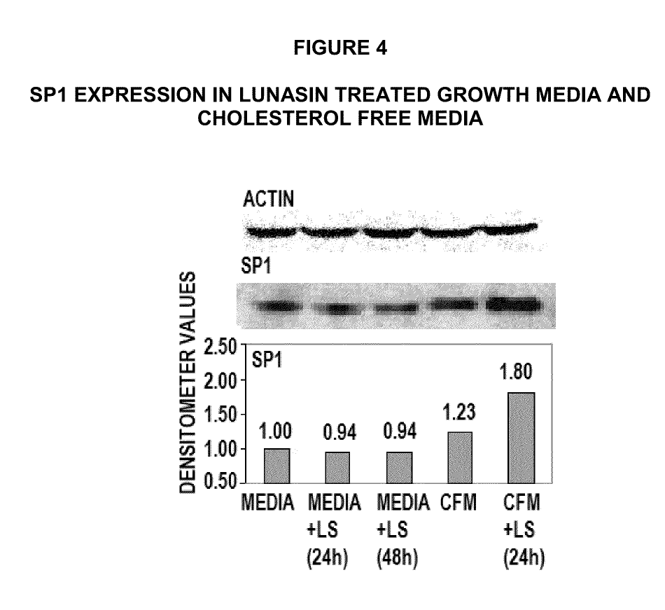

Unlike HMG-CoA reductase, SREBP activation of LDL-receptor by sterol depletion requires increased recruitment of Sp1 co-activator to a site adjacent to SREBP in the promoter/regulatory sequence of LDL-receptor gene (25). As shown in FIG. 3, the up regulation of LDL-receptor by lunasin (LS) in cholesterol-free media may be due to increased availability and recruitment of the Sp1 coactivator to the LDL-receptor promoter/regulatory sequence. To test this hypothesis, the level of Sp1 was determined in lunasin-treated growth media and cholesterol-free media by Western analysis using Sp1 antibody, as follows: HepG2 cells (1×10⁶) were grown from confluence in DMEM with 10% FBS for 24 hours before growth media was replaced with fresh growth media or cholesterol-free media (to activate SREBP) and treated with, or without 10 uM synthetic lunasin. After 24 hours, total protein was extracted from each treatment and 10 ug protein loaded onto 10% Tris-glycine gels, electroblotted onto nitrocellulose membrane, and immunostained with primary antibodies raised against Sp1 and actin (to show equal loading of proteins). Spot densitometer values were obtained by digital scanning and Un-Scan It software and represent data from one experiment. The results are shown in FIG. 4.

FIG. 4 shows that Sp1 levels in control and lunasin-treated growth media were not significantly different. However, Sp1 levels increased in cholesterol-free media by 23%, compared to the growth media. The addition of lunasin in the cholesterol-free media further increased Sp1 levels by almost 60%, which closely mirrors the increase in LDL-receptor levels in lunasin-treated, cholesterol-free media.

The data from these experiments indicate that the increase in LDL-receptor expression by lunasin in sterol-depleted media could be attributed to the increased availability of the Sp1 transcriptional co-activator. Also, the inhibition of HMG-CoA reductase expression by lunasin lowers intracellular cholesterol levels that keep SREBP activated, resulting in the upregulation of LDL receptor expression.

Therefore, the data shows that lunasin inhibits the expression of HMG-CoA reductase, the rate limiting enzyme in the body's metabolic pathway for synthesis of cholesterol, and at the same time increases the expression of the LDL receptor, leading to increased clearance of low-density lipoprotein (LDL) from the bloodstream, which will lower total and LDL cholesterol in a mammal.

Most circulating cholesterol in mammals is synthesized internally, on average 1000 mg/day compared to 200-300 mg/day from intestinal intake in a human diet. Thus the internal production of cholesterol, as catalyzed by HMG-CoA reductase and the amount of LDL receptors in liver cell membranes, is the single most important factor in modulating cholesterol levels in mammals. Accordingly, these experiments demonstrate that an effective amount of lunasin reduces both LDL and total cholesterol levels in a mammal.

### Example 3

Lunasin can be extracted from commercial sources of soy protein. Lunasin has been found in significant amounts from commercial sources of soy protein and its homologues from other seed sources such as barley and wheat. To identify preferred sources for the starting raw material that can be used for lunasin extraction, several commercially available soy protein products were screened for the presence of lunasin.

The procedure used was as follows: approximately 500 mg of soy protein samples obtained from different commercial sources (Solae, St. Louis, MO) were dissolved in 50 mL of aqueous phosphate buffer (pH 7.2) by shaking for 1 hour at room temperature. Samples were centrifuged at 2500 rpm for 30 minutes and the aqueous fraction separated and put in separate tubes. Protein concentrations were measured by Bradford assay and around 20 ug of total protein were loaded onto two Bio-Rad Laboratories (Hercules, Calif.) 16% Tris-Tricine gels. One of the SDS-PAGE gels (I) was stained with Coomasie blue and destained before digital imaging. The 5 kDa lunasin band is indicated by arrow. The other (II) is electroblotted onto nitrocellulose membrane and incubated with affinity-purified lunasin polyclonal antibody (Pacific Immunology, Ramona, Calif.) followed by HRP-conjugated donkey anti-rabbit secondary antibody (Amersham Biosciences, Piscataway, N.J.). Lunasin immunosignals (indicated by arrow) are detected using the ECL Western blotting kit from Amersham.

The results showed that lunasin concentration varies dramatically from source to source. This assay is a useful tool in identifying sources of natural lunasin for use in the compositions and methods of the present invention. The soy concentrate that contained the most lunasin was used as a starting material in a buffer extraction procedure to produce the lunasin-enriched soy concentrate (LeSC) used in the following experiments.

### Example 4

Formulated lunasin-enriched soy concentrate (LeSC) and LeSC supplemented with soy flour (SF) contain significant amounts of lunasin. This experiment evaluated the amount of lunasin in lunasin-enriched soy concentrate (LeSC) and LeSC supplemented with soy flour.

Lunasin-enriched soy concentrate was produced by first identifying commercially available soy protein preparations that contain significant amounts of lunasin by Western blot analysis using lunasin polyclonal antibody, as described in Example 3. The soy protein concentrate identified to contain the most lunasin was used as starting material in a one-step buffer extraction procedure (0.1× PBS pH 7.2) followed by centrifugation to separate the supernatant. Two volumes of acetone were added to supernatant and precipitate was separated by centrifugation with filter bags before vacuum drying to get the lunasin-enriched soy concentrate.

Efforts to make lunasin more resistant to undesired excessive digestion, improve its bioavailability, and retain its bioactivity when ingested, resulted in the discovery of at least one of the preferred embodiments of the present invention, a composition comprising lunasin enriched soy concentrate and soy flour.

In at least one embodiment of the present invention, compositions of the present invention that comprise naturally derived lunasin can be optimized for use in particular methods of the present invention by varying the amount of total protein and lunasin content, which can be controlled by the amount of soy concentrate used, and varying the amount of lunasin protection from digestion, which can be controlled by the amount of minimally heated soy flour used.

For food based items it is sometimes desirable to limit the amount of protease inhibitors in a product. For example, U.S. Patent Application No. 20070092633, filed Apr. 26, 2007, hereby incorporated by reference, teaches that part of the standard processing of some soy products includes heat treatment to inactivate anti-nutritional elements such as Bowman-Birk and Kuntz inhibitors. Therefore, in a preferred embodiment of the present invention, a composition comprising lunasin and soy flour is optimized through preparation methods describe herein or known to one skilled in the art, to have a level of protease inhibitors sufficient to protect lunasin biological activity during digestion but not sufficient to have levels of anti-nutritional elements that are undesirable for oral use.

Clinical trials on a 50:50 blend of soy concentrate and soy flour led to a 20-30% reduction of LDL cholesterol (17, 18.) Those clinical trials were performed without the knowledge that lunasin is an active element in soy concentrate in reducing LDL cholesterol, and therefore did not control for the level of lunasin present in the blend. The present invention teaches improved methods of determining lunasin concentration in starting materials and final products of the present invention, so as to maximize the concentration of lunasin and therefore the activity of compositions of treatment in cholesterol related applications. In at least one preferred embodiment of the present invention the ratio of soy flour to soy concentrate is between 10:90 and 50:50, more preferably between 20:80 and 40:60, more preferably approximately 30:70 soy flour:soy concentrate. This ratio for minimally heated soy flour and soy concentrate was determined to provide a biologically active concentration of lunasin and as well as sufficient protection from digestion by the soy flour.

In the following several experiments, minimally heated soy flour (SF) was added to the starting soy concentrate (at a 30:70 w/w mixture) before buffer extraction with 0.1× PBS pH 7.2 and acetone precipitation to produce lunasin enriched soy concentrate plus soy flour (LeSC+SF.)

The Western blotting analysis procedure used in this experiment was as follows: approximately 20 ug of total protein from LeSC, SF and the LeSC+SF were electrophoresed in 16% Tris-Tricine gels and electroblotted onto nitrocellulosemembrane. Blots were incubated with lunasin polylconal antibody followed by HRP-conjugated anti-rabbit secondary antibody before lunasin immunosignals were detected with the ECL kit. The results showed that both LeSC and LeSC+SF contained significant amounts of lunasin.

### Example 5

Lunasin-enriched soy concentrate with soy flour (LeSC+SF) retains bioactivity even when digested with digestive enzymes. Biological activity of LeSC (A), LeSC+SF (B), digested LeSC+SF (C), digested LeSC (D), digested soy protein isolate (E) and digested soy concentrate (F) was measured using the H3 histone acetyltransferase (HAT) assay (see Example 8.) Around 100 mg total protein of LeSC, LeSC+SF, soy protein isolate and soy concentrate were digested by mixing pancreatin (Sigma Life Sciences, Saint Louis, Mo.) at 1:1 (w/w) and incubating for 30 min. at 40° C. To confirm that the HAT assay is working, treatment with synthetic lunasin (+synL) was included. Synthetic lunasin reduced acetylation of histone H3 by the histone acetylase enzyme, PCAF, using core histones isolated from chicken erythrocyte (Upstate/Millipore, Billerica, Mass.) as template for the HAT assay. Around 10 ug of sample protein was incubated with 1 ug of core histones before undergoing HAT reaction with PCAF enzyme and acetyl CoA substrate. Reaction products were run on 16% Tris-Tricine gels and electroblotted onto nitrocellulose membrane. Blots were incubated with primary antibody raised against acetylated H3 (diacetylated at histone14 and histone10) and HRP-conjugated anti-rabbit secondary antibody before detecting signals using the ECL kit. Low signals indicated that the lunasin peptide was bioactive because it prevented the acetylation of histone H3. Strong signals indicated that the lunasin peptide had been digested and rendered inactive, thus failing to impact levels of histone H3 acetylation.

There was significant reduction in H3 acetylation in the presence of synthetic lunasin compared to the untreated control. Both the LeSC and the LeSC+SF were able to significantly reduce H3 acetylation by PCAF, indicating that the lunasin found in both soy protein extracts is biologically active. Pancreatin digestion of LeSC+SF reduced the biological activity but not to the extent observed when LeSC alone is digested. Like LeSC, soy protein isolate and soy concentrate that contain significant amounts of lunasin, did not show lunasin biological activity after pancreatin digestion These results indicate that the formulated LeSC+SF protects lunasin to a certain degree from pancreatin digestion, and allows lunasin to retain its biological activity.

### Example 6

Partial digestion of formulated LeSC+SF increases biological activity of lunasin. A confirmatory experiment to determine the biological activity of digested and undigested LeSC and LeSC+SF was conducted using a different core histone template. This time we used the core histones extracted from HeLa tumor cells. Unlike the chicken erythrocyte cells, core histones from sodium butyrate treated HeLa cells are commercially available (Upstate/Millipore, Piscataway, N.J.), and can be used as a positive control for histone acetylation. The core histones isolated from untreated HeLa cells were used as a negative control (low levels of histone acetylation) and as template for the HAT assay.

The HAT bioactivity assay was conducted using acid extracted core histones from HeLa cells (Upstate/Millipore) as a template (temp (−) control) for the PCAF catalyzed HAT reaction. Core histones from sodium butyrate (NaB) treated HeLa cells were used as a positive control since NaB is a histone deacetylase inhibitor known to increase histone acetylation. The inhibitory effect of synthetic lunasin (+synL) on histone H3 acetylation by PCAF was used to compare the effect of lunasin-enriched soy concentrate (A), digested LeSC (A dig), LeSC+SF (B) and digested LeSC+SF (B dig). LeSC and LeSC+SF were partially digested by adding pancreatin at 1:0.5 (w/w) and incubating at 38° C. for 15 min. The numbers below the legend indicate relative densitometer readings normalized using immunosignal from the template (temp). Low numbers indicate presence of lunasin biological activity.

The results showed that significant reduction in H3 acetylation in the presence of synthetic lunasin was seen. The undigested LeSC (A) and LeSC+SF (B) showed reduced levels of H3 acetylation, indicating that the natural lunasin found in these soy extracts was biologically active. Partial digestion of LeSC (A Dig) led to the loss of biological activity.

Surprisingly, partial digestion of LeSC+SF resulted in an increase in biological activity rather than a decrease. While it is not intended that the present invention be limited to any precise mechanism, it is believed that lunasin is covalently bound to high molecular weight protein complexes and that, with the protection of soy flour, partial digestion only breaks down these bonds and releases, but does not destroy, bioactive lunasin into the solution. In a preferred embodiment of the present invention, lunasin is partially digested prior to use. In another preferred embodiment of the present invention, soy flour is present when lunasin is partially digested.

LeSC+SF was partially digested by mixing it with freshly prepared pancreatin solution (10 μg/mL of distilled water) in a 1:0.5, (w/w) ratio. Mixture was incubated at 38° C. for 15 min. before proteases and digestive enzymes were inactivated by boiling for 5 min and then quenching in ice. Under these digestion conditions the lunasin in the LeSC soy extract was digested and inactivated while that of LeSC+SF were more biologically active. However, the conditions for the partial digestion of LeSC+SF has to be determined empirically by analyzing digestion products for lunasin content and biological activity using the HAT assay.

Variations in the sources of pancreatin and protease enzymes, the age of the protease enzyme, or incubation conditions can lead to variability in digestion conditions. For example, the use of one month old preparations of pancreatin for partial digestion led to the degradation and loss of activity of lunasin under similar incubation conditions described above. Therefore, in a preferred embodiment of the present invention, acceptable ranges for concentration of and incubation time with the protease enzymes are determined using an assay such as the HAT assay used above to evaluate biological activity of the treated compositions.

### Example 7

Chymotrypsin inhibitors (Chy) protect the bioactivity of lunasin. To determine which protease inhibitors found in soy protects lunasin from digestion, soybean trypsin inhibitor and trypsin+chymotrypsin inhibitors were obtained from Sigma and mixed with LeSC on 1:1 w/w ratio. The mixtures were digested with pancreatin, and digestion products immunostained with lunasin antibody.

Details of the experiment are as follows. LeSC+soybean trypsin inhibitors (1:1 w/w) (Sigma) and LeSC+trypsin and chymotrypsin inhibitors (1:1 w/w) (Sigma) were digested with pancreatin (1:1 w/w) by incubating at 38° C. for 15 min. Digestion products and LeSC were analyzed by Western blot analysis using lunasin primary antibody and synthetic lunasin as standard controls.

HAT bioactivity assay was conducted using core histones from chicken erythrocyte cells (Upstate/Millipore) as a template for the PCAF catalyzed HAT reaction. The inhibitory effect of synthetic lunasin (+synL) on histone H3 acetylation by PCAF as compared to the negative untreated control (−synL) was used to compare the effect of digested LeSC (A), digested LeSC+try+chy (B), digested LeSC+try (C), undigested LeSC (D) and undigested LeSC +SF (E.)

The results showed that in the LeSC+trypsin+chymotrypsin inhibitors sample lunasin was better protected from digestion than in the LeSC+trypsin inhibitor sample. Likewise, in HAT assays to determine lunasin biological activity, digestion of LeSC+trypsin+chymotrypsin inhibitors was significantly more bioactive than LeSC+trypsin inhibitor. Pancreatin digestion of LeSC led to the loss of biological activity. These results indicate that the presence of chymotrypsin inhibitors in lunasin-enriched soy concentrate (LeSC) both helps protect the biological activity of lunasin and helps protect lunasin from excessive digestion.

### Example 8

Screening Assay to Determine Lunasin Biological Activity.

Core histones purified from chicken erythrocyte cells were used as templates in histone acetylase (HAT) reactions using PCAF histone acetylase enzyme, in the presence or absence of around 2-10 uM lunasin. The core histone template and lunasin-enriched soy concentrates (LeSC and LeSC+SF) were mixed (10:1 w/w) and incubated in ice for 5 min and 25° C. for 10 min before mixture was added to 1× HAT reaction mix, 1 uM acetyl CoA and 5 uL PCAF (based on recommended concentration from Upstate/Millipore). Reaction mixture was incubated at 30° C. while shaking at 250 rpm for 1 h. Reaction was stopped by adding Laemmli stop buffer (1:1 v/v) with beta-mercaptoethanol, and boiling for 5 min. before quenching in ice for 15 min. The products of PCAF HAT reaction were run on 16% SDS-PAGE, blotted onto nitrocellulose membrane and immunostained with primary antibodies raised against diacetylated histone H3 (Ac-Lys 13+Ac-Lys14 H3) followed by HRP-conjugated anti-rabbit secondary antibody. Chemiluminescent signals from antibody complexes were visualized using standard chemiluminescent reagents and exposed to Kodak BioMAX film, developed and spot densitometer measured by using digital scanner and UN-SCAN-IT software program from Silk Scientific (Orem, Utah).

The results showed the reduction of H3 acetylation in the reaction mixtures treated with LeSc and LeSC+SF as compared to the untreated control, indicating that this screening procedure can determine the biological activity of lunasin-enriched soy concentrates. It was also determined that digestion of LeSC eliminates biological activity but not that of LeSC+SF which shows only a partial reduction of biological activity.

### Example 9

The in vivo activity of the presently described compositions, as well as treatment utilization of kits and treatment methods, may be optionally determined by either of the following procedures.

Male dogs (beagles, ranging from about 9 to about 14 kilograms, 1 to 4 years old) are fed a standard dog feed supplemented with 5.5% lard and 1% cholesterol. Baseline blood samples are drawn from fasted dogs prior to initiating the study to obtain reference values for plasma cholesterol. Dogs are then randomized to groups of five animals with similar plasma cholesterol levels. The animals are dosed in accordance with a treatment method described herein immediately prior to diet presentation for seven days. Blood samples are obtained 24 hours after the last dose for plasma cholesterol determinations. Plasma cholesterol levels are determined by a modification of the cholesterol oxidase method using a commercially available kit.

In an optional alternative procedure, hamsters are separated into groups of six and given a controlled cholesterol diet containing 0.5% cholesterol for seven days. Diet consumption is monitored to determine dietary cholesterol exposure. The animals are dosed in accordance with a treatment method described herein once daily beginning with the initiation of diet. Dosing is by oral gavage. All animals moribund or in poor physical condition are euthanized. After seven days, the animals are anesthetized by intramuscular (IM) injection of ketamine and sacrificed by decapitation. Blood is collected into vacutainer tubes containing EDTA for plasma lipid analysis and the liver is excised for tissue lipid analysis. Lipid analysis is conducted as per published procedures (e.g., Schnitzer-Polokoff et al., Comp. Biochem. Physiol., 99A, 4 (1991), pp. 665-670 and data is recorded as percent reduction of lipid versus control.

### REFERENCES

The numeric references incorporated above in parentheses correspond to the following list of published papers and abstracts. All of the below listed publications are herein incorporated by reference in their entirety to the same extent as if each individual publication was specifically and individually indicated to be incorporated by reference in its entirety.
1. Galvez, A. F., Revilleza, M. J. R. & de Lumen, B. O. A novel methionine-rich protein from soybean cotyledon: cloning and characterization of cDNA. Plant Physiol 114:1567 (1997).
2. Galvez, A. F. & de Lumen, B. O. A soybean cDNA encoding a chromatin-binding peptide inhibits mitosis of mammalian cells. Nature Biotech. 17: 495-500 (1999).
3. de Mejia E G, Vasconez M., de Lumen B O & Nelson R. Lunasin concentration in different soybean genotypes, commercial soy protein and isoflavone products. J Agric Food Chem 52: 5882-5887 (2004).
4. Galvez, A. F. Chen, N., Macasieb, J., & de Lumen, B. O. Chemopreventive property of a soybean peptide. Cancer Res. 61:7473-7478 (2001).
5. De Pinho, R. A. The cancer-chromatin connection. Nature 391: 533-536 (1998).
6. Kuzmin I & Geil L. DNA methylation and chromatin modifications in cancer and development. Int Arch Biosci 2001: 1047-1056 (2001).
7. Magbanua M, Dawson K, Huang L, Malyj W, Gregg J, Galvez A & Rodriguez R L. Nutrient-Gene Interactions Involving Soy Peptide and Chemopreventive Genes in Prostate Epithelial Cells, in Nutritional Genomics—Discovering the Path to Personalized Nutrition, J. Kaput and R. L. Rodriguez eds., Wiley and Sons, New Jersey (2005).
8. Bennett M K & Osborne T F. Nutrient regulation of gene expression by the sterol regulatory element binding proteins: Increased recruitment of gene-specific coregulatory factors and selective hyperacetylation of histone H3 in vivo. PNAS 97: 6340-6344 (2000).
9. Brown M S & Goldstein J L. Lowering plasma cholesterol by raising LDL receptors. Atherosclerosis Suppl 5: 57-59 (2004).
10. Sirtori C R, Gatti E, Mantero O, Conti F., et al. Clinical experience with the soybean protein diet in the treatment of hypercholesterolemia. Am J Clin Nutr. 32:1645-1658 (1979).
11. Descovich G C, Ceredi C., Gaddi A., Benassi M S, et al., Multicentre study of soybean protein diet for outpatient hyper-cholesterolaemic patients. Lancet 2:709-712 (1980).
12. Lam, Y., Galvez, A., and de Lumen, B. O. Lunasin suppresses EIA-mediated transformation of mammalian cells but does not inhibit growth of immortalized and established cancer cell lines. Nutrition & Cancer, 47: 88-94 (2003).
13. Coqueret, O. New roles for p21 and p27 cell-cycle inhibitors: A function for each cell compartment? Trends in Cell Biology, 13: 65-70, (2003).
14. Bruzzone, R., White, T. W., and Paul, D. L. Connections with connexins: The molecular basis of direct intercellular signaling. European Journal of Biochemistry, 238: 1-27 (1996).
15. Mullen E, Brown R M, Osborne T F & Shay N F. Soy isoflavones affect sterol regulatory element binding proteins (SREBPs) and SREBP-regulated genes in HepG2 cells. J. Nutr. 134: 2942-2947 (2004).
16. Gherardi E., Thomas K, Le Cras T D, Fitzsimmons C, Moorby C D & Bowyer D E. Growth requirements and expression of LDL receptor and HMG-CoA reductase in HepG2 hepatoblastoma cells cultured in a chemically defined medium. J Cell Sci. 103:531-539 (1992).
17. Sirtori C R, Gatti E, Mantero O, Conti F., et al. Clinical experience with the soybean protein diet in the treatment of hypercholesterolemia. Am J Clin Nutr. 32:1645-1658 (1979).
18. Descovich G C, Ceredi C., Gaddi A., Benassi M S, et al., Multicentre study of soybean protein diet for outpatient hyper-cholesterolaemic patients. Lancet 2:709-712 (1980).
19. Yu Y, Teng Y, Liu H, Reed S H & Waters T. UV irradiation stimulates histone acetylation and chromatin remodeling at repressed yeast locus. PNAS 102:8650-8655 (2005).
20. Kim M-K, Shin J-M, Eun H C & Chung J H., The role of p300 histone acetyltransferase in UV-induced histone modifications and MMP-1 gene transcription. PloS One 4: e4864 (2009).

I claim:

1. A method for protecting against photoaging of skin in an individual, comprising: (a) providing: (i) an individual desiring to prevent photoaging of skin and, (ii) a composition comprising a compound selected from the group consisting of the peptide of SEQ ID NO 2 and a functionally equivalent variant, fragment or analogue of said peptide; and (b) administering said composition to said subject to protect against photoaging.

2. The method of claim 1, wherein said compound is obtained from soybean, wheat or barley.

3. The method of claim 1, wherein said compound is obtained by producing, extracting and purifying said compound using recombinant DNA techniques.

4. The method of claim 1, wherein said compound is obtained by synthetic polypeptide production.

5. The method of claim 1, wherein said individual is a human.

6. The method of claim 1 wherein administering comprises topical administration of the composition.

7. The method of claim 1, wherein the composition is in the form of a semi-solid formulation, liquid, gel, suspension, or aerosol spray.

8. The method of claim 1, wherein said composition further comprises chymotrypsin inhibitor.

9. The method of claim 1, wherein said compound is administered to said individual at between 5 μg/ml and 50 μg/ml.

10. A method for protecting against collagen breakdown in the skin of an individual, comprising: (a) providing: (i) an individual desiring to prevent collagen breakdown in the skin and, (ii) a composition comprising a compound selected from the group consisting of the peptide of SEQ ID NO 2 and a functionally equivalent variant, fragment or analogue of said peptide; and (b) administering said composition to said subject to protect against collagen breakdown.

11. The method of claim 10, wherein said compound is obtained from soybean, wheat or barley.

12. The method of claim 10, wherein said compound is obtained by producing, extracting and purifying said compound using recombinant DNA techniques.

13. The method of claim 10, wherein said compound is obtained by synthetic polypeptide production.

14. The method of claim 10, wherein said individual is a human.

15. The method of claim 10 wherein administering comprises topical administration of the composition.

16. The method of claim 10, wherein the composition is in the form of a semi-solid formulation, liquid, gel, suspension, or aerosol spray.

17. The method of claim 10, wherein said composition further comprises chymotrypsin inhibitor.

18. The method of claim 10, wherein said compound is administered to said individual at between 5 μg/ml and 50 μg/ml.

19. A method for protecting against wrinkling of the skin in an individual, comprising: (a) providing: (i) an individual desiring to prevent wrinkling of skin and, (ii) a composition comprising a compound selected from the group consisting of the peptide of SEQ ID NO 2 and a functionally equivalent variant, fragment or analogue of said peptide; and (b) administering said composition to said subject to protect against wrinkling of the skin.

20. The method of claim 19, wherein said compound is obtained from soybean, wheat or barley.

21. The method of claim 19, wherein said compound is obtained by producing, extracting and purifying said compound using recombinant DNA techniques.

22. The method of claim 19, wherein said compound is obtained by synthetic polypeptide production.

23. The method of claim 19, wherein said individual is a human.

24. The method of claim 19 wherein administering comprises topical administration of the composition.

25. The method of claim 19, wherein the composition is in the form of a semi-solid formulation, liquid, gel, suspension, or aerosol spray.

26. The method of claim 19, wherein said composition further comprises chymotrypsin inhibitor.

27. The method of claim 19, wherein said compound is administered to said individual at between 5 μg/ml and 50 μg/ml.


SEQUENCE LISTING (2 sequences)

SEQ ID NO: 1 - protein, 157 aa, Glycine max
MTKFTILLISLLFCIAHTCSASKWQHQQDSCRKQLQGVNLTPCEKHIMEKIQGRGDDDDDDDDDNHILRTMGGRINYIRRNEGKDEDEEEGHMQKCCTEMSELRSPKCQCKALQKIMENQSEELEEKQKKKMEKELINLATMCRFGPMIQCDLSSDD

SEQ ID NO: 2 - protein, 43 aa, Glycine max
SKWQHQQDSCRKQLQGVNLTPCEKHIMEKIQGRGDDDDDDDDD